(19) United States
(12) Patent Application Publication
(10) Pub. No.: US 2012/0150281 A1
(43) Pub. Date: Jun. 14, 2012

(54) IMPLANT MADE OF BIOCORRODIBLE MATERIAL AND WITH A COATING CONTAINING A TISSUE ADHESIVE

(76) Inventors: Bjoern Klocke, Zuerich (CH); Matthias Gratz, Erlangen (DE)

(21) Appl. No.: 13/312,017

(22) Filed: Dec. 6, 2011

Related U.S. Application Data
(60) Provisional application No. 61/420,808, filed on Dec. 8, 2010.

(51) Int. Cl. A61F 2/82

(52) U.S. Cl. 623/1.34; 623/1.46; 623/1.42

(57) ABSTRACT

The invention relates to an implant having a base body made of a biocorrodible material. A surface of the implant has a coating at least in some regions, wherein the coating comprises a tissue adhesive.

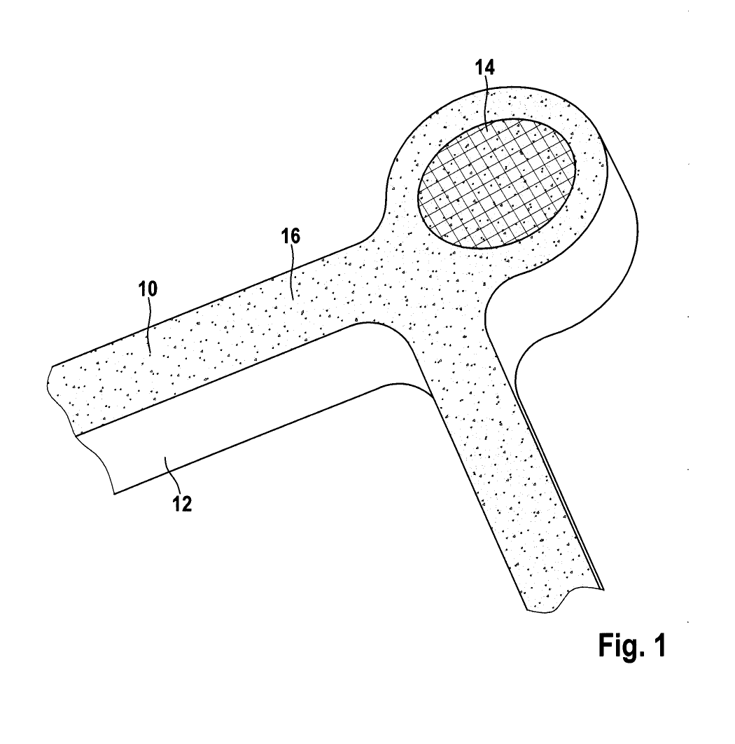

### CROSS REFERENCE

The present application claims priority on U.S. Provisional Application No. 61/420,808 filed on Dec. 8, 2010; which application is incorporated herein by reference.

### TECHNICAL FIELD

The invention relates to a coated implant having a base body made of a biocorrodible material.

### BACKGROUND

Implants are used in a variety of different embodiments in modern medical technology. They serve, for example, for supporting vessels, hollow organs and systems (endovascular implants, e.g. stents), for fastening and temporarily fixing tissue implants and tissue transplantations, for orthopedic purposes, for example as nail, plate or screw, and for other applications. A particularly frequently used form of an implant is the stent.

The implantation of stents has established itself as one of the most effective therapeutic measures for treating vascular diseases. Stents have the purpose to perform a support function in the hollow organs of a patient. For this purpose, some stents have a filigree support structure of metallic struts which, for inserting into the body, has initially a compressed form and expands at the site of the application. One of the main fields of application of such stents is to permanently or temporarily expand or keep open vascoconstrictions, in particular constrictions (stenoses) of the coronary vessels. Besides that, also known are, for example, aneurism stents which primarily serve for sealing the aneurism. The support function is provided additionally.

Many stents have a circumferential wall with a sufficient bearing capacity to keep the constricted vessel open to a desired extent, and a tubular base body through which the blood can pass unhindered. The circumferential wall is usually formed by a grid-like support structure which allows to insert the stent in a compressed state with small outer diameter up to the constriction to be treated of the respective vessel and to expand the stent there, for example, with a balloon catheter, wide enough that the vessel has the desired enlarged inner diameter. Alternatively, shape-memory materials such as nitinol have the ability of self-expansion once a reset force is gone which keeps the implant at a small diameter. The reset force is usually exerted on the material by a protective hose.

The implant, in particular the stent, has a base body made of an implant material. An implant material is a non-living material which is used for applications in medicine and interacts with biological systems. Basic requirement for the use of a material as implant material which, during the intended purpose, is in contact with the body environment is the body compatibility (biocompatibility) of the material. Biocompatibility is to be understood as the ability of a material to generate in a specific application, an appropriate tissue reaction. This includes the adaptation of the chemical, physical, biological and morphological surface properties of an implant to the host tissue with the goal of a clinically desired interaction. The biocompatibility depends further on the temporal progress of the reaction of the biosystem into which the material is implanted. For instance, relatively short-term irritations and inflammations occur which can result in tissue changes. Accordingly, depending on the properties of the implant material, biological systems react in various ways. Corresponding to the reaction of the biosystem, the implant materials can be divided in bioactive, bioinert, and degradable/absorbable (herein referred to as biocorrodible) materials.

Implant materials comprise polymers, metallic materials and ceramic materials (for example as coating). Biocompatible metals and metal alloys for permanent implants include, for example, stainless steels (for example 316L), cobalt-based alloys (for example CoCrMo-casting alloys, CoCrMo-forge alloys, CoCrWNi-forge alloys and CoCrNiMo-forge alloys), pure titanium and titanium alloys (e.g. cp titanium, TiAl6V4 or Ti-Al6Nb7) and gold alloys. In the field of biocorrodible stents, the use of magnesium or pure iron as well as biocorrodible base alloys of the elements magnesium, iron, zinc, molybdenum and wolfram is proposed.

After the implantation of biodegradable implants, in particular stents, there is the risk that incompletely degraded fragments or, in some cases, non-degradable components such as X-ray markers, can trigger undesired consecutive reactions. In case of a stent, said fragments, for example, can be carried along in the blood flow and can cause embolisms. This is in particular the case if the stent is not yet sufficiently grown into the vessel wall. Specifically in case of implants with an antiproliferative coating, this problem can occur more frequently.

Degradable stents made from magnesium alloy, but also from polymeric materials, are usually not X-ray visible. Therefore, X-ray markers should be attached to the stent. The simplest markers are permanent markers from gold or tantalum which—optionally electrically insulated—are attached to the stent. However, after the degradation of the magnesium stent, said markers are no longer mechanically pressed against the vessel wall by the stent. There is the risk that the marker becomes detached in the blood flow (lumen) and also causes embolisms distally in the respective artery.

For the mentioned clinical and regulatory reasons, as well as others, potentially occurring fragments of the biocorrodible implant (coming from base body, marker or other additional elements with degradation properties different from the ones of the base body) pose potential problems and challenges. Specifically in case of active ingredient-coated degradable stents where the use of antiproliferative medicaments delays or disturbs the healing process, the risk of fragment generation is increased.

### SUMMARY

One or more of the aforementioned disadvantages of the prior art are solved or at least minimized by implant embodiments according to the invention having a base body made of a biocorrodible material. A surface of the implant has a coating at least in some regions, wherein the coating comprises or consists entirely of a tissue adhesive.

At least some aspects of the invention is based on the discovery that by using a medical tissue adhesive, the base body and/or other additional elements having degradation properties different from the ones of the base body can be securely bonded to the tissue at the implantation site. For this purpose, the tissue adhesive can be applied on the whole surface of the implant or only on sections of the surface of the implant.

### DESCRIPTION OF THE DRAWINGS

The invention is illustrated in more detail hereinafter by means of an exemplary embodiment and an associated FIGURE. The single FIGURE shows a schematic illustration of a stent having the coating according to the invention.

### DETAILED DESCRIPTION

Turning now to a more detailed description of various invention embodiments, as tissue adhesive useful for invention embodiments, many different medical tissue adhesives are suitable. In some embodiments, the tissue adhesive is a cyanoacrylic acid ester adhesive, in others a fibrin adhesive, and in other embodiments other adhesives.

The effect of cyanoacrylic acid-based adhesives is based on the polymerization of cyanoacrylic acid monomers into polymeric and/or copolymeric structures.

The cyanoacrylic acid ester adhesives preferred for the solution according to some invention embodiments are self-biodegradable over the course of one or more days, in other embodiments in one or more weeks, and in still other embodiments in a month. Other time periods will be useful in other embodiments.

Thus, for the formation of suitable cyanoacrylic acid ester adhesives, amongst others, copolymers from the following cyanoacrylate monomers and non-cyanoacrylate monomers are suitable: Cyanoacrylate monomers: Alkyl cyanoacrylates, alkenyl cyanoacrylates, alkoxyalkyl cyanoacrylates; non-cyanoacrylate monomers: Glycolyds, lactides and caprolactones.

Other monomers are also considered as being particularly useful according to some invention embodiment as long as they are suitable for forming biodegradable polymeric structures.

Furthermore, the cyanoacrylic acid ester adhesive is preferably present as a polymer of cyanoacrylate monomers.

In one example embodiment, particularly useful for implant fixation at the implantation site of non-degradable components of at least partially degradable implants, the use of non-biodegradable or only very slowly biodegradable cyanoacrylic acid ester adhesives in the meaning of this invention is also suitable, e.g., cyanoacrylate polymers from monomers with long organic chains such as, e.g., octyl cyanoacrylates or others. In this embodiment, the adhesive material as well as the non-degradable implant components remains at the implantation site.

Suitable fibrin adhesives in the context of the present invention are two-component systems which, when mixing the components, act promptly and are strongly adhesion-promoting. For the use in coatings, the ingredients known to the person skilled in art in many embodiments are not present in premixed form. The coating according to many invention embodiments provides one of the two components of the tissue adhesive. The usability of fibrin adhesives is based here on the fact that the fibrinogen, which is usually contained in one of the components of the fibrin adhesive, in contrast to the embodiments known to the person skilled in the art, is not part of a coating according to these invention embodiments because, due to the use of the implant in blood vessels, it is already present at the implantation site. In one embodiment of the coating according to the invention, aprotinin together with the other usual constituents of fibrin adhesives, including factor XIII, thrombin and a calcium source such as, e.g., calcium chloride is present. Only by the recruitment of circulating fibrinogen after the implantation, the adhesion is ensured. Put another way, the tissue adhesive is formed at the implantation site after the coating contacts circulating fibrinogen.

A coating in the meaning of the invention is an application, at least in sections, of the components of the coating onto the base body, X-ray marker and/or further structural elements of the implant. Preferably, in case of a stent, the coating is applied only abluminally on the implant. A layer thickness in some embodiments is in the range of 1 nm to 100 μm, in other embodiments from 300 nm to 15 μm, and in other embodiments other thicknesses, including less than 1 nm or more than 15 μm. The coating according to the invention can be applied directly onto the implant surface, or further intermediate layers are provided; thus, if necessary, the base body of the implant can have an inorganic base layer which improves the adhesion of the coating according to the invention. Onto the coating according to the invention, further layers can be applied, for example, to facilitate the insertion of the implant into the body. Methods for coating implants are well known to the person skilled in the art and include, for example, pipetting, rolling up, spraying, dipping and others.

Designated as biocorrodible in the meaning of the invention are materials in which in a physiological environment, a degradation/conversion takes place so that the part of the implant consisting of said material is no longer present or at least a predominant portion is not present anymore.

Preferably, the biocorrodible implant material is a biocorrodible metal alloy or a biodegradable polymer.

Preferably, the biocorrodible implant material is a biocorrodible magnesium alloy, an iron alloy or a biocorrodible polyactide, in particular L-polyactide (PLLA). A magnesium alloy is to be understood here as a metallic microstructure, the main component of which is magnesium. Main component is the alloy component with the highest weight proportion of the alloy. A proportion of the main component is more than 50% by weight in some embodiments, more than 70% by weight in other embodiments, and other portions in other embodiments. The composition of the alloy is to be selected in such a manner that it is biocorrodible. As test medium for testing the corrosion behavior of a potential alloy, an artificial plasma can be used as it is specified according to EN ISO 10993-15:2000 for biocorrosion studies (composition NACl 6.8 g/l, CaCl₂ 0.2 g/l, KCl 0.4 g/l, MgSO₄ 0.1 g/l, NaHCO₃ 2.2 g/l, Na₂HPO₄ 0.126 g/l, NaH₂PO₄ 0.026 g/l). For this purpose, a sample of the alloy to be tested is stored in a sample container with a defined quantity of the test medium at 37° C. and pH 7.38. In time intervals—adapted to the corrosion behavior to be expected—of a few hours up to several months, the samples are removed and are checked in a known manner for signs of corrosion. The artificial plasma according to EN ISO 10993-15:2000 corresponds to a medium similar to blood and thus represents a possibility to reproducibly simulate a physiological environment in the meaning of the invention.

In a particular embodiment, the implant comprises additional elements such as, for example, a permanent or biodegradable X-ray marker. Suitable X-ray markers are known in the prior art and can be produced, among other things, on the basis of chemical elements with high atomic numbers such as tantalum, or ionic or non-ionic complexes, as well as on the basis of polymers containing, e.g. iodine or gadolinium. For example, a permanent marker can be fixed on a base body of a biocorrodible implant. Subsequently, the coating according to the invention can be applied onto said implant. Preferably, the coating containing the tissue adhesive is applied only onto the X-ray marker. Furthermore, the permanent X-ray marker is preferably coated with a non-biodegradable cyanoacrylic acid adhesive. Said devices have the advantage that the adhesive material as well as the non-degradable implant components remains at the implantation site. Thus, side effects caused by non-degradable elements of the implant are prevented.

The coating can contain one or more active ingredients which are released after the implantation. An active ingredient in the meaning according to the invention is to be understood as a drug having a pharmaceutical effect which serves for healing, relief, prevention or diagnosis of diseases in the human or animal body. Active ingredients comprise in particular paclitaxel, sirolimus and derivates of the latter. Particularly advantageous are active ingredients which act on mTOR as well as RAS inhibitors, in particular such inhibitors which prevent the RAS adhesion.

The part of the coating containing the active ingredient does not have to be identical to the layer which contains the tissue adhesive. Thus, the active ingredient-containing layer can be applied in addition the tissue adhesive. Said layer can be applied below or above the tissue adhesive. In still other embodiments, different layers can be applied side by side—a first layer containing an active ingredient on one portion of the base body and a second layer containing the tissue adhesive on a second portion of the base body (with neither of the first and second layers overlaying the other).

In a particular embodiment, at least one layer, preferably abluminal layer, for example for facilitating the endothelialization, is provided.

In a further embodiment, the active ingredient-containing layer is located below the tissue adhesive and directly on the implant surface. Said active ingredient layer thus represents an adhesion promoter (primer) for the tissue adhesive on the implant surface. For this, the active ingredient-containing coating in some embodiments is additionally roughened or is provided with symmetrical or random structural elements such as recesses, indentations, (flutes, grooves) projections or similar structural features to enhance adhesion.

For some embodiments in which it is desirable to achieve enhanced adhesion of the tissue adhesive on the implant surface, the surface of the implant is additionally subjected to a mechanical and/or chemical preparation. For instance, an adhesion promoter (primer), e.g., silanes, preferably a polymeric adhesion promoter such as, e.g. parylenes, are coated onto the implant surface. Alternatively or additionally, in some embodiments the surface of the implant is roughened or in other embodiments is provided with symmetrical or random structural elements such as recesses, indentations, (flutes, grooves) projections or similar structural features to enhance adhesions.

In order to prevent abrasion or activation of the coating containing the tissue adhesive during insertion of the implant, for example, in some embodiments a coating material having a high layer adhesion or a temporary topcoat is applied. Alternatively, in still other embodiments abrasion or activation is avoided by providing a sleeve or protective hose that contains the implant and which is retracted directly before placing the implant to thereby expose the implant only when it is at the implant site. When using active ingredient-eluting systems, in particular the active ingredient-containing layer can serve as temporary topcoat for the layer containing the tissue adhesive that decomposes to expose the adhesive layer. For this, in some embodiments the surface of the topcoat is additionally roughened or is provided with symmetrical or random structural elements such as recesses, indentations, (flutes, grooves) projections or similar structural features to increase adhesion.

In order to increase the storage stability of such a coating according to the invention, upon completion of the coating and until use, in many embodiments the implant is protected against contact with aqueous media and high humidity. For this purpose, “dry” protective gases (with an example being nitrogen) as well as moisture-tight packages, for example on the basis of aluminum or plastic, are used.

Implants in the meaning of the invention are devices inserted into the body by a surgical method. At least some implants comprise fastening elements for bones, for example, screws, plates or nails, surgical sutures, intestinal staples, vascular clips, prostheses in the region of the hard and soft tissue, and anchor elements for electrodes, in particular of pacemakers or defibrillators. The implant of invention embodiments comprises or consists of the biocorrodible material. Preferably, the implant is a stent.

The above and further example elements of some invention embodiments are illustrated in the attached FIGURE. The single FIGURE illustrates a stent 10 having a base body 12 made of a biocorrodible magnesium alloy and an X-ray marker 14 fixed on the base body 12. On the abluminal side of the base body 12 as well as on the X-ray marker, a coating 16 (indicated by dotting) is applied which contains a tissue adhesive, for example, a cyanoacrylic acid ester adhesive. The X-ray marker 14 has different degradation properties than the base body 12, with the result that the base body 12 may degrade before the X-ray marker 14 does 14. In the prior art, the X-ray marker may have thus been released to the blood stream upon degradation of the base body 12. Through the invention embodiment, however, this is avoided as the coating 16 binds the X-ray marker 14 and base body 12 to the tissue.

It will be apparent to those skilled in the art that numerous modifications and variations of the described examples and embodiments are possible in light of the above teaching. The disclosed examples and embodiments are presented for purposes of illustration only. Therefore, it is the intent to cover all such modifications and alternate embodiments as may come within the true scope of this invention.

What is claimed is:

1. An implant having a base body comprising a biocorrodible material, wherein a surface of the base body has a coating at least in some regions, which coating comprises a tissue adhesive.

2. The implant according to claim 1, wherein the tissue adhesive is one or more of a cyanoacrylic acid ester adhesive or a fibrin adhesive.

3. The implant according to claim 1, wherein the implant has an X-ray marker which carries the coating at least in some regions and wherein the X-ray marker has different degradation properties than the base body.

4. The implant according to claim 1, wherein the coating further comprises at least one active ingredient.

5. The implant according to claim 1, and further comprising a second coating comprising an active ingredient, the second coating applied one of below or above the tissue adhesive coating.

6. The implant according to claim 5, wherein the active ingredient is one or more of paclitaxel, sirolimus or a derivate of sirolimus.

7. The implant according to claim 5, wherein the second active ingredient coating is applied abluminally on the implant.

8. The implant according to claim 1, and further comprising an adhesion promoter for the tissue adhesive coated on the implant surface.

9. The implant according to claim 1, wherein the implant surface is configured for enhanced adhesion with the coating through one of being roughened or being provided with symmetrical or random structural elements including one or more of recesses, flute shaped indentations, grooves, or projections.

10. The implant according to claim 1, and further comprising a temporary topcoat.

11. The implant according to claim 1, and further comprising one of a removable sleeve or a removable protective hose containing the implant and coating and configured to be removed to expose the implant and coating at the implantation location.

12. The implant according to claim 1, wherein the base body comprises a biocorrodible magnesium alloy.

13. The implant according to claim 1, wherein the implant is a stent.

14. The implant according to claim 1, wherein the base body has degradation properties, and further comprising an X-ray marker that has slower degradation properties than the base body wherein the base body will degrade before the X-ray marker, and wherein the coating is applied to at least portions of both the base body and the X-ray marker wherein the X-ray marker will be bound to tissue after the base body decomposes.

15. The implant according to claim 1 wherein the tissue adhesive is cyanoacrylic acid ester adhesive made from octyl cyanoacrylate monomer.

16. An implant according to claim 1 wherein the tissue adhesive is a fibrin adhesive that is formed at the implantation site after the coating contacts circulating fibrinogen.

17. An implant according to claim 1 wherein the tissue adhesive is a fibrin adhesive wherein the coating comprises aprotinin, factor XIII, thrombin, and a calcium source, and wherein the fibrin adhesive is formed at the implantation site after the coating contacts circulating fibrinogen.

18. An implant according to claim 1 and further comprising:
an inorganic base layer between the coating and the base body, the inorganic layer enhancing adhesion of the coating to the base body; and,
wherein the coating has a thickness of between about 300 nm to 15 μm.

19. An implant according to claim 1 and further comprising:
a temporary topcoat layer over the coating and comprising an active ingredient, the temporary topcoat layer decomposing after the implant is at the implantation site to expose the coating; and
wherein the base body is biocorrodible and comprised of L-polyactide.

20. A stent comprising:
a biocorrodible base body made of a magnesium alloy;
an X-ray marker having degradation characteristics that are different from those of the base body;
at least one coating comprising a tissue adhesive and an active ingredient, the tissue adhesive being a fibrin adhesive that is formed at the implantation site after the coating contacts circulating fibrinogen, the coating having a thickness of between about 300 nm to 15 μm, the coating covering at least a portion of the base body and at least a portion of the X-ray marker and useful to bind both the base body and the X-ray marker to tissue at an implant location; and,
wherein the at least one coating is protected until the stent is at the implantation site by one of a removable sleeve or at least topcoat.
